(19) United States
(12) Patent Application Publication
(10) Pub. No.: US 2020/0001498 A1
(43) Pub. Date: Jan. 2, 2020

(54) TISSUE SUBSTITUTE PRINTING

(71) Applicant: regenHU AG, Villaz-St-Pierre (CH)

(72) Inventors: Michael Kuster, Liebefeld (CH); Marc Thurner, Wavre (CH)

(73) Assignee: regenHU AG, Villaz-St-Pierre (CH)

(21) Appl. No.: 16/486,527

(22) PCT Filed: Feb. 13, 2018
(86) PCT No.: PCT/EP2018/053519

§ 371 Date: Aug. 16, 2019

(30) Foreign Application Priority Data
Feb. 22, 2017 (CH) 00201/17

(51) Int. Cl. B28B 1/00; A61L 27/12; C04B 35/447

(52) U.S. Cl. CPC B28B 1/001; B33Y 30/00; C04B 35/447; A61L 27/12

(57) ABSTRACT

A cartridge for use in a tissue substitute printing system is disclosed, the cartridge may have:
a material reservoir, the material reservoir storing a tissue substitute material in a flowable, in particular paste-like form;
a material aperture arranged at a bottom side of the cartridge, the material aperture being fluidic coupled with the material reservoir for releasing tissue substitute material stored in the material reservoir;
at least one auxiliary aperture arranged at the bottom side of the cartridge for releasing an auxiliary medium, the at least one auxiliary aperture being arranged in proximity to and fluidic separate from the material aperture.

A tissue substitute printing system and a method for manufacturing a tissue substitute are also disclosed.

### TECHNICAL FIELD

The present disclosure is related to the field of manufacture of tissue substitutes. It is particularly related to cartridges for use in a tissue substitute printing system, to tissue substitute printing systems, as well as manufacturing methods for tissue substitutes.

### BACKGROUND

It is known to repair tissue defects, in particular bone defects, by inserting a tissue substitute material into a tissue cavity, in particular a bone cavity, in a paste-like form. The tissue substitute material may be prepared in its paste-like form either individually by mixing of a powder-type cement material with a liquid solvent, or may be readily provided in its paste-like form in a syringe-like cartridge. After insertion respectively injection into the tissue cavity, in particular bone cavity, setting of the tissue substitute material (cement material) is achieved by the solvent being displaced by water that is comprised e. g. in blood, bone marrow, or Ringer's solution. A corresponding commercial product is available as VELOX® where the paste-like bone substitute material is calcium phosphate based bone cement (CPC), mixed with an anhydrous oil.

### SUMMARY OF DISCLOSURE

In many applications, it would be desirable to provide and insert a tissue implant or tissue substitute directly with the final three-dimensional geometry. This, however, is critical because of the paste-like and non-solid properties of some tissue substitute materials in their processable form.

It is an overall objective of the present invention to improve the situation regarding the manufacture of tissue substitutes or tissue implants. Favorably, in particular the manufacture of tissue substitutes or tissue implants from paste-like tissue substitute materials is improved. In a general way, the overall objective is achieved by the subject matter of the independent claims. Exemplary and favorable embodiments are further defined by the dependent claims and the overall disclosure of the present document.

In an aspect, the overall objective is achieved by a cartridge for use in a tissue substitute printing system. The cartridge comprises a material reservoir, the material reservoir storing a tissue substitute material in a flowable, in particular paste-like form. The cartridge further includes a material aperture arranged at a bottom side of the cartridge. The material aperture is fluidic coupled with the material reservoir for releasing tissue substitute material stored in the material reservoir. The material aperture is typically a single aperture. The cartridge further includes at least one auxiliary aperture arranged at the bottom side of the cartridge for releasing an auxiliary medium. The at least one auxiliary aperture is arranged in proximity to and fluidic separate from the material aperture.

In application, tissue substitute material is released in a controlled and metered manner via the material aperture onto a target. Further, auxiliary medium is released onto the target via the at least one auxiliary medium aperture simultaneously or alternating with the tissue substitute material. On the target, the tissue substitute material and the auxiliary medium react. By simultaneously spatially displacing the target and the cartridge with respect to each other in accordance with a pre-determined tissue substitute geometry, a tissue substitute or tissue implant of the predetermined tissue substitute geometry is generated.

The bottom side of the cartridge is a side of the cartridge that faces the target. In a typical orientation during application, the orientation of gravity is from top to bottom, with the target being arranged below the cartridge.

In combination, the tissue substitute material and the auxiliary medium form a two-component system. The auxiliary medium is a curing agent for the flowable tissue substitute material that cures or sets the tissue substitute material respectively supports the curing or setting after application onto the target by way of a chemical reaction and/or a physical interaction between the components. In the uncured, i. e. flowable and typically paste-like form, the tissue substitute material has sufficient geometric stability to maintain its shape when applied in small volumes in worm-like or drop-like form onto the target. In dependence of the specific situation and application, a further post-printing curing or hardening may be carried out subsequent to the printing in order to tune the biological and/or mechanical properties of the tissue substitute.

In some typical embodiments, the cartridge is a single-use cartridge that stores an amount of tissue substitute material that is required for manufacturing a single tissue substitute or implant. Subsequently, the cartridge is typically discarded.

In dependence of the specific application and the tissue substitute that is manufactured, the material reservoir may store between typically 0.5 cc (0.5 ml) and 30 cc (30 ml) of tissue substitute material.

The material aperture has a typical diameter in a range of 0.1 mm to 5 mm, for example 1 mm. However, other and in particular larger diameters may be chosen as necessary. The at least one auxiliary aperture has a typical diameter in a range of e. g. 0.05 mm to 0.5 mm.

The cartridge may be manufactured form one or more of a variety of materials, such as metal, glass or plastics. In dependence of the material or materials used and the specific cartridge design, a variety of manufacturing technologies, such as injection moulding, conventional machining, etching, or laser-ablation, may be employed. Favourably, elements that come in contact with the tissue substitute material respectively the auxiliary medium are made from a material that is inherent (does not react) with respect to the tissue substitute material respectively auxiliary medium, or corresponding contact surfaces are coated with such material.

In some embodiments, the tissue substitute material comprises calcium phosphate. It may especially be a calcium phosphate paste. Calcium phosphate is particularly suited for the manufacture of bone substituting implants. However, other materials in flowable and in particular paste-like form, such as hydraulic cements or self-setting cements may also be used. The tissue substitute material may for example comprise bioactives such as, e.g., bone morphogenic proteins and ions, e. g. strontium ions, magnesium or silica particles, and/or one or more of natural and synthetic hydrogels, such as Colagen. The tissue substitute material may optionally further comprise drugs, such as antibiotics.

The auxiliary medium is a fluid, in particular a liquid or a gas In some embodiments, the auxiliary medium is or comprises at least one of water, an aqueous solution, a body fluid, cell culture liquid, Ringer's solution, or a natural or synthetic hydrogel. A typical example of a suited gaseous auxiliary medium is water vapour respectively steam. Generally the auxiliary medium is selected in dependence of the tissue substitute material that shall be cured respectively settled or for which curing or setting shall be improved. Selection of the auxiliary medium may further be based on manufacturing environmental conditions, such sterility requirements. In case the auxiliary medium is a body fluid, it is favourably a body fluid, e. g. blood, of the patient for whom the tissue substitute is produced.

As will be explained in more detail further below by way of various embodiments, the cartridge is designed such that the tissue substitute material and the auxiliary medium are separate and do not come into contact with each other. For this purpose, the fluidic system of the tissue substitute material and the auxiliary medium are distinct and separate.

In some embodiments, the cartridge comprises an auxiliary medium reservoir. The auxiliary medium reservoir stores an auxiliary medium. The auxiliary medium reservoir is fluidic coupled with the at least one auxiliary aperture. Both the tissue substitute material and the auxiliary medium are stored by the cartridge. Further, a fluidic system in the form of flow channels, ductworks or the like is integrated into the cartridge in order to fluidic couple the material aperture with the material reservoir and the at least one auxiliary aperture with the auxiliary medium reservoir. Therefore, a cartridge according to this type of embodiment is particularly simple and convenient in use, without exposing or requiring the separate handling of the tissue substitute material and/or auxiliary medium. The auxiliary medium reservoir is distinct from and fluidic uncoupled with respect to the material reservoir.

The auxiliary medium may be a liquid or gas as explained above. It is provided in a volume that at least corresponds to respectively is sufficient for hardening the tissue substitute material, typically with some surplus or safety margin.

In some embodiments with an auxiliary medium reservoir, the auxiliary medium reservoir at least partially surrounds the material reservoir. Such embodiment is particularly compact and favourable in application. The material reservoir and the auxiliary medium reservoir may both have, e. g., a circular cross section (cross section traverse to a longitudinal cartridge axis), with the auxiliary medium reservoir circumferentially surrounding the material reservoir in concentric arrangement. The material reservoir and the auxiliary medium reservoir may, e. g., be barrel-respectively cylindrically shaped and have a generally closed bottom surface where the material aperture and the at least one auxiliary aperture are arranged.

In some embodiments, the cartridge comprises a plurality of auxiliary apertures. A plurality of auxiliary apertures via which auxiliary medium is released simultaneously respectively in parallel may be used to ensure uniform contact of the tissue substitute material, thereby ensuring uniform contact and hardening. In further embodiments as explained further below, auxiliary medium release may be controlled separately for different auxiliary apertures. In such embodiments, one or more apertures may be selected for the auxiliary medium release in dependence of the relative motion direction between cartridge and target.

In some embodiments with a plurality of auxiliary apertures, the auxiliary apertures are arranged along an arc around the material aperture. The arc may for example be a circular arc that is concentric with the material aperture. The arc may be closed, resulting in the material aperture being surrounded by auxiliary apertures. In other embodiments, however, a number of e. g. three auxiliary apertures is arranged on an arc segment of, e. g. totally 90 degrees to 120 degrees.

In some embodiments with a plurality of auxiliary apertures, the cartridge further comprises a liquid distribution ductwork. The auxiliary medium distribution ductworks fluidic couples with the plurality of auxiliary apertures. Such embodiment may be favourably used for simultaneous release of auxiliary medium via a number or all auxiliary apertures. The liquid distribution network distributes the auxiliary medium form a common source to the auxiliary apertures. The source may be an auxiliary medium reservoir of the cartridge as explained above, an inlet opening that receives auxiliary medium form a source external to the cartridge as explained further below, or a combination of both.

In further embodiments, only a single auxiliary aperture is present and arranged in proximity to the material aperture. In still further embodiments, an auxiliary aperture is of annular or arc shape and circumferentially surrounds the material aperture fully or partly.

In some embodiments, the cartridge comprises at least one inlet opening. The at least one inlet opening is fluidic coupled with the at least one auxiliary aperture. The inlet opening serves for receiving auxiliary medium from a source external to the cartridge, such as a separate auxiliary medium cartridge, a pouch, a syringe, a container, or the like. The fluidic coupling is realized by a corresponding flow channel or a system of flow channels, such as a distribution ductwork as explained before.

Optionally, the inlet opening may be arranged for mounting of a fluidic fitting and may therefore, e. g. have a corresponding fit or thread, typically an inner thread. Further, the cartridge may comprise a fluidic fitting, such as a tube fitting, a bayonet fitting or a Luerlock fitting which proves or opens into the inlet opening. In further variants, tubing may be part of or be fixed to the cartridge, the tubing opening into the at least one inlet opening.

In some embodiments, at least one auxiliary aperture is fluidic coupled with an associated inlet opening via a point-to-point coupling. The fluidic coupling is realized via a corresponding flow channel of the cartridge. Because of the point-to-point coupling, the flow channel is unbranded and leads directly from the inlet opening to the auxiliary aperture. Favourably, the flow channel is as short as possible in accordance with the cartridge design and may, e. g. be a straight flow channel or be made from two or more straight segments.

In embodiments with a plurality of auxiliary apertures, a single inlet opening may be present and the auxiliary medium may be distributed from the single inlet opening to the auxiliary apertures via a distribution ductwork. In further embodiments with a plurality of auxiliary apertures, each auxiliary aperture is fluidic coupled with an associated inlet opening via a point-to-point coupling. The number of inlet openings accordingly corresponds to the number of auxiliary apertures. Such embodiment may be favourably used for individually controlling the auxiliary medium release via the single auxiliary apertures. In further embodiments, a plurality of auxiliary apertures is divided into a number of sub-groups, with a number of. e. g. two or three auxiliary apertures belonging to a sub-group. An associated inlet opening may be present for each sub-group.

In some embodiments, the cartridge comprises an inner cartridge element and an outer cartridge element. The outer cartridge element is arranged circumferentially around the inner cartridge element. The inner cartridge element forms the material reservoir. A flow channel in fluidic coupling with the at least one auxiliary aperture may be present and be arranged in an interface region between the inner cartridge element and the outer cartridge element.

The outer cartridge element of such embodiment forms a shell into which the inner cartridge element is inserted. The inner cartridge element and the outer cartridge element may be assembled e. g. via a press-fit, gluing, or plastic welding, or screwing. Both the inner cartridge element and the outer cartridge element may have an e. g. cylindrical overall shape with a typically conical or frustum-conical bottom section. The material aperture is arranged at a bottom side, in particular a bottom wall, of the inner cartridge element. One or more auxiliary apertures may be arranged at a bottom side, in particular a bottom wall, of the outer cartridge element.

One or more inlet openings may be arranged in the circumferential wall of the outer cartridge element. In some embodiments, the outer cartridge element receives the inner cartridge element without a gap being present between them. The one or more inlet opening(s) may be fluidic coupled with the at least one auxiliary aperture via a flow channel that is arranged at the interface between the inner cartridge element and the outer cartridge element. Such flow channel may be realized by a channel on the inner surface of the circumferential wall of the outer cartridge element, a cannel on the outer surface of the inner cartridge element, or both. The inner surface of the circumferential wall of the outer cartridge element and the outer surface of the circumferential wall of the inner cartridge element face each other. In a further embodiment, a flow channel or auxiliary medium distribution ductwork is arranged fully within the circumferential wall of the outer cartridge element between its inner and its outer surface.

In particular in embodiments with an inlet opening in fluidic coupling with a plurality of auxiliary apertures, a circumferential gap may be present between the inner cartridge element and the outer cartridge element. The gap forms a continuous fluidic room that fluidic couples with the inlet opening and the auxiliary apertures, thereby coupling the inlet opening with the auxiliary apertures.

In further embodiments, a volume is present between the outer cartridge element and the inner cartridge element in form of an annular space that serves as auxiliary fluid reservoir.

In some embodiments, the cartridge includes a temperature controller. The temperature controller is arranged for controlling a temperature of the tissue substitute material in the material reservoir. Controlling the temperature of the tissue substitute material may be favourable to in order to control the rheological properties, such as consistency, viscosity, and ductility of the tissue substitute material in its flowable, paste-like form.

In some embodiments, the temperature controller is or comprises an electric resistive heating element that is, in turn powered, and control by the tissue substitute printing system.

Instead of a pure heating element, a Peletier element may be used which may be controlled for alternatively heating or cooling. In a further embodiment where the cartridge is fully or partly made from a heat-conductive material, in particular metal, the cartridge material itself, e. g. a. material of the material reservoir, may serve as temperature controller that is, in operation, thermally coupled with a heating and/or cooling element, such as an electric resistive heating element or a Peletier element. Such element may integrated into the cartridge receptacle of a tissue substitute printing system as explained further below.

In further variants, the temperature controller is formed by a heating and/or cooling fluid flow channel in thermal coupling with the tissue substitute material. The heating and/or cooling fluid flow channel has a corresponding inlet and outlet that are arranged to fluidic couple with a heating/and or cooling system of a tissue substitute printing system. In operation, a liquid or gaseous heating and/or cooling fluid is passed through the heating and/or cooling fluid flow channel, thereby heating or cooling the tissue substitute material as needed.

In some embodiments, the material aperture is a bore that is arranged in a cartridge bottom wall or a hollow needle element that projects beyond a cartridge bottom. While a bore as material aperture is particularly favourable for manufacture, a hollow needle may be advantageous in view of a controlled and well-defined geometry of the released material and in particular in order to prevent smearing of the typically paste-like material and a contamination of the cartridge. Typically, a needle extends by e. g. 0.1 mm to 2 mm below the bottom surface and towards the target.

In some embodiments, the cartridge further includes a material release device. The material release device may, in some embodiments, be comprise a material release piston that is sealing displaceable arranged in the material reservoir. In operation, the material release piston is displaced in a controlled way, thereby, reducing the material-filled inner volume of the material reservoir in a syringe-like way and forcing tissue substitute material out of the material aperture. The displacement is typically a linear displacement towards the bottom of the cartridge and in particular of the material reservoir. In operation, displacement of the material release piston is controlled via a material release controller of a tissue substitute printing system as explained further below, e. g. hydraulically pneumatically or via coupling engagement with a linear displacement drive. If the material release piston is displaced hydraulically or pneumatically, the cartridge may include a corresponding pneumatic or hydraulic coupler. If the material release piston is displaced mechanically, the material release piston may be designed for a pushing engagement to receive a pushing force from a linear displacement drive of the material release controller. Alternatively, the material release device may include a spindle drive as part of the cartridge for displacing the material release piston. In such embodiment, the spindle drive receives a rotational driving torque from the material release controller which in this case includes a rotatory drive. In further embodiments, a linear drive includes a coil, similar to a voice coil, that is arranged displace, upon being energized, the material release piston magnetically In further embodiments, material release is achieved via a controlled overpressure supply, e. g. gas pressure supply, that is configured to pressurize the tissue substitute material inside the material reservoir for a controlled respectively metered release out of the material reservoir. In such embodiment, no material release piston may be required. The overpressure supply of such embodiments, is typically part of the tissue substitute printing system and the material release device may be realized as fluidic coupler in fluidic coupling with the material reservoir.

In embodiments where the cartridge includes an auxiliary medium reservoir, the cartridge may include an auxiliary medium release device. The auxiliary medium release device is arranged for releasing auxiliary medium out of the auxiliary medium reservoir via the at least one auxiliary aperture. The auxiliary medium release device may be designed in generally the same way and work according as a material release device. In operation, release of auxiliary material is controlled via an operative coupling of the auxiliary medium release device with an auxiliary medium release controller of a tissue substitute printing system. The auxiliary medium release device may also be a fluidic coupler in fluidic coupling with the auxiliary medium reservoir for applying an over pressure to the auxiliary medium, thereby pressurizing the auxiliary medium inside the auxiliary medium reservoir and pneumatically forcing auxiliary medium out of the auxiliary medium reservoir via the at least one auxiliary aperture.

According to a further aspect, the overall objective is achieved by a tissue substitute printing system. The tissue substitute printing system includes a cartridge receptacle. The cartridge receptacle is configured to replaceable receive a cartridge as disclosed above and/or further below. The system further includes a target receptacle. The system further includes a kinematic structure. The kinematic structure is configured to spatially displace the cartridge receptacle and the target receptacle relative to each other. The system further includes a material release controller. The material release controller is designed to control release of tissue substitute material via the material aperture. The system further includes an auxiliary medium release controller. The auxiliary medium release controller is designed to control release of auxiliary medium via the at least one auxiliary aperture. The system further includes a computerized control unit. The control unit is configured to control operation of the material release controller and the auxiliary medium release controller for a metered release of tissue substitute material and a simultaneous or alternating release of auxiliary medium. The control unit is further configured to simultaneously control the kinematic structure to displace the cartridge receptacle and the target receptacle relative to each other in accordance with a pre-determined tissue substitute geometry.

The cartridge receptacle typically includes the required interfaces that are designed for coupling the medium release controller with the medium release device of the cartridge and, if desired, for coupling the auxiliary material release controller with an auxiliary medium release device of the cartridge.

The target receptacle is designed to receive the target on which the tissue substitute or implant is formed. Typically, the target receptacle includes a support platform and a positioning device in order to ensure a defined geometric arrangement of target and cartridge. In some embodiments, the target receptacle may include fixation or clamping devices for the target. The target receptacle is arranged to maintain the target in a horizontal or leveled orientation with respect to gravity. Further with respect to gravity, the target receptacle is generally arranged below the cartridge receptacle, such that, in operation, the target is located below the cartridge. In some embodiments, the target receptacle includes a platform on which the target is placed.

The kinematic structure comprises actuators that are controlled in operation. The kinematic structure may be Cartesian structure as generally known in the art, with three mutually orthogonal axes that are individually controlled. The axes may be distributed between the cartridge receptacle and the target receptacle. In an embodiment, either of the target receptacle and the cartridge receptacle is fixed relative to a support structure of the tissue substitute printing system, while the other is displaced in three axes. In another variant, the target receptacle is displaced in two lateral axes (horizontal respectively perpendicular to gravity), while the cartridge receptacle is displaced vertically, i. e. along the direction of gravity, or vice versa. In a further embodiment, the cartridge receptacle is displaced along one horizontal axis and the vertical axis, while the target receptacle is displaced along the other horizontal axis. Further movements or degrees of freedom with corresponding actuators, such as rotation around the vertical axis, may be optionally present. In further embodiments, another type of kinematic structure, such as a SCARA structure, is used.

The material release controller may, for example, be designed as linear displacement drive with a corresponding actuator, e. g. a motor. The linear displacement drive is designed to couple to the material release device, such as a material release piston of a cartridge, as explained above. The material release controller may include a plunger rod or threaded spindle that is designed to couple to the material release piston. In case the material release device of the cartridge comprises a treaded spindle as explained before, the material release controller may be designed as rotatory drive. In further embodiments, the material release controller includes a gas power generator, in particular an air power generator, such as a compressor to pneumatically pressurize the tissue substitute material directly or via a material release piston as explained before.

The material release controller may optionally further include a material release sensing device that is designed and arranged to determine an amount of released tissue substitute material. The material release sensing device, may, e. g., be a rotatory encoder on a motor axis of the actuator. In a further embodiment, the material release sensing device is a linear encoder that measures linear displacement of a threaded spindle or piston rod.

In embodiments where the cartridge includes an auxiliary medium reservoir as explained before, the auxiliary medium release controller may be designed in generally the same way as the material release controller. For example, the auxiliary medium release controller may include a gas power generator, in particular an air power generator, such as a compressor to pneumatically pressurize the auxiliary medium.

In further embodiments, the material release controller and/or the auxiliary medium release controller may include one or more corresponding control valves, e. g. proportional vales and/or switching valves for controlling the supply of gas pressure for pressurizing the tissue substitute material and/or the auxiliary medium.

In some embodiments, pressurized gas, such as pressurized air, may be provided by an external source, such as a gas cartridge or a pressurized air supply.

In designs where the cartridge does not include an auxiliary medium reservoir, the tissue substitute printing system includes an auxiliary medium reservoir receptacle or is designed to couple to an auxiliary medium reservoir, such as a gas cartridge or an external gas supply.

In embodiments where the auxiliary medium is provided in a syringe-like cartridge with a piston or in a syringe, the auxiliary medium release controller may include a corresponding spindle drive to force auxiliary medium out of the auxiliary medium reservoir. In further embodiments, the auxiliary medium release controller includes a controlled pump, e. g. liquid pump, to provide liquid to the cartridge. In further embodiments, the auxiliary medium is provided pre-pressurized e. g. from an external supply or from an elevated auxiliary medium reservoir, and the auxiliary medium release controller includes a control valve to control supply to the cartridge.

In embodiments where the cartridge includes a plurality of auxiliary medium apertures that may be controlled separately, the auxiliary medium release controller may include a number of functionally independent sub-units, each being associated with a corresponding auxiliary medium aperture or set of auxiliary medium apertures. In such embodiments, the subunits may be controlled separately.

The computerized control unit is typically based on a computer and runs a corresponding program code. The computer hardware may, in some embodiment, be based on a general-purpose computer, such as a personal computer or workstation. The control unit may further include interface and/or power circuitry for interfacing and controlling the further units as explained before, or such interface and/or power circuitry may be part of such units.

In embodiments where the cartridge includes a temperature controller as explained before, the tissue substitute printing system favourably includes a temperature controller as explained before. Operation of the temperature controller is controlled by the control unit.

The tissue substitute geometry is favourably provided in form of a numeric data set, such as CAD/CAM data. The data may be provided on a data carrier, such as CD Rom or hard disk, for which the control unit may include a corresponding interface and/or reading device. Further, the control unit may be designed to receive the data via a data interface such as LAN or WLAN interface.

The control device is favourably designed to control the manufacture of the tissue substitute in a number of layers that are arranged on above the other. Between the layers, the target receptacle with the target and the cartridge carrier with the cartridge are favourably vertically displayed by the layer thickness which may, e. g. be in a range of 0.1 mm to 1 mm.

While releasing the tissue substitute material and auxiliary medium for an individual layer, the target receptacle with the target and the cartridge receptacle with the cartridge are displaced horizontally with respect to each other as required by the tissue replacement geometry in the corresponding layer. During manufacture of an individual layer, tissue substitute material is favourably not released continuously but tissue release may be activated and stopped via the material release controller. The same may hold true for the control of auxiliary medium release via the auxiliary medium release controller.

The tissue substitute material is favourably released in the form of material elements in form of droplets and/or in string- or worm-like form. The material elements may contact each other or be separate from each other.

For curing respectively setting the tissue substitute material, auxiliary medium is released by corresponding control of the auxiliary medium release controller either continuously along with releasing tissue substitute material. Alternatively, tissue substitute material and auxiliary material may be released in an alternating manner.

For a release of tissue substitute material and auxiliary medium in an alternating manner, it may be switched between the release of tissue substitute material and auxiliary medium in intervals that are sufficiently short respectively with a frequency that is sufficiently high to allow full curing respectively hardening of the tissue substitute material directly after printing, e. g. within 1 sec. to 10 sec. after exiting the material release aperture.

In further embodiments where the tissue substitute is manufactured in layers, the control device may control the release of tissue substitute material only, thereby printing a single layer or a number of e. g. two or three layers of tissue substitute material, and subsequently release auxiliary medium only to cure the previously printed layer or layers. In such embodiment, the at least one auxiliary aperture may be realized as one or more spray nozzles that ensure a distribution of the released auxiliary medium over a larger area in a uniform or substantially uniform way. While the path or trajectory of relative movement between target and cartridge respectively material aperture is, for each layer, determined by the tissue substitute geometry when releasing tissue substitute material, the relative movement between target and cartridge respectively auxiliary apertures is favourably determined such that the whole lateral area of the tissue substitute is covered by the released auxiliary medium, while following the path of the tissue substitute geometry is not required. For tissue substitutes of comparatively small lateral dimensions and/or a sufficiently large spray angle, no relative movement may be required at all and the cartridge respectively the at least one auxiliary aperture may be positioned above a centre area of the carrier for releasing the auxiliary medium.

In some further embodiments, the tissue substitute printing system includes an additional curing device, such as an infrared (IR) and/or ultraviolet (UV) or visible light source and/or a heating device under control of the control unit. A method for manufacturing a tissue substitute may include curing respectively setting released tissue substitute material by exposing it with radiation and/or over temperature. Such embodiment may be favourable e. g. where the auxiliary medium is a photocrosslinkable hydrogel, such as a phPEG-based with e. g. EO-SIN Y or Irgacure as photo inhibitor.

According to a still further aspect, the overall objective is achieved by a method for manufacturing a tissue substitute. The method includes releasing tissue substitute material in a flowable, in particular paste-like form from a cartridge in a controlled and metered manner onto a target. The method further includes simultaneously or alternatingly releasing an auxiliary medium from the cartridge, such that the material and the auxiliary medium react on the target. The method further includes simultaneously spatially displacing the target and the cartridge with respect to each other in accordance with a pre-determined tissue substitute geometry.

According to a still further aspect the overall objective is achieved by a tissue substitute manufacturing kit. The tissue substitute manufacturing kit includes a tissue substitute printing system and a cartridge in accordance with the present disclosure. A tissue substitute manufacturing kit may further include an auxiliary medium reservoir with auxiliary medium.

An embodiment of the method may be carried out with a cartridge according to any embodiment and a corresponding tissue substitute printing system. Therefore, disclosed embodiments of a cartridge respectively tissue substitute printing system also disclose a corresponding method embodiment and vice versa.

### BRIEF DESCRIPTION OF THE DRAWINGS

FIG. 1a-1g: illustrates schematically a first embodiment

FIG. 2a-2g: illustrates schematically a second embodiment;

FIG. 3a-3g: illustrates schematically a third embodiment;

FIG. 4a-4e: illustrates schematically a fourth embodiment;

FIG. 5a-5e: illustrates schematically a fifth embodiment;

### EXEMPLARY EMBODIMENTS

The figures are shown to schematically illustrate the five embodiments but do not depict all the structural details of the cartridge system.

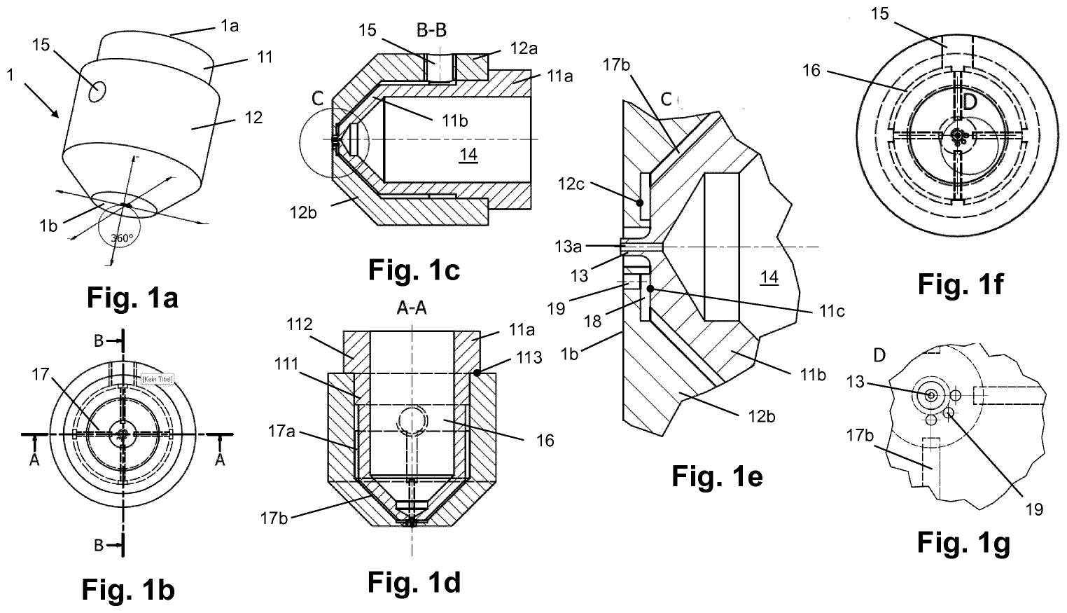

FIGS. 1a, to 1g show an embodiment of a cartridge 1 that is designed in accordance with the present disclosure. FIG. 1a shows a perspective view, FIG. 1b a top view and FIG. 1f a bottom view. FIG. 1c, and FIG. 1d are cross sectional views B-B and A-A as indicated in FIG. 1b. FIG. 1e shows detail C as indicated in FIG. 1c and FIG. 1g shows detail D as indicated in FIG. 1f.

The cartridge 1 with a cartridge top 1a and a cartridge bottom 1b comprises an inner cartridge element 11 and an outer cartridge element 12 in coaxial arrangement. The inner cartridge element 11 has a tubular inner cartridge main section 11a that merges into a frustum-conical or conical inner cartridge bottom section 11b. Similarly, the outer cartridge element 12 has a tubular outer cartridge main section 12a that merges into a frustum-conical outer cartridge bottom section 12b. The inner cartridge element 11 fits into the outer cartridge element 12 as explained further below. The inner volume of the inner cartridge element 11 forms the material reservoir 14 that is filled with e. g. a paste-like calcium phosphate composition.

As best visible in FIG. 1e, the inner cartridge bottom section 11b merges at its bottom side into a nozzle 13 in fluidic coupling with the material reservoir 14. The bottom opening of the nozzle 13 forms the material aperture 13a. The nozzle 13 slightly projects beyond the cartridge bottom 1b, which however, is not essential.

The bottom section 11b of the inner cartridge element 11 that is enclosed by the outer cartridge element has an outer contour that generally corresponds to the inner contour of the outer cartridge element 12, with the inner cartridge element 11 and the outer cartridge element 12 being dimensioned to establish a tight fit. The inner cartridge element 11 however, has a top portion 112 of increased diameter, thereby forming a shoulder 113 which abuts against the top rim of the outer cartridge element 12, thereby axially positioning the inner cartridge element 11 with respect to the outer cartridge element 12.

An inlet opening in form of a radial through-bore 15 is present in the wall of the outer cartridge 12 respectively its main section 12a. The bore 15 is exemplarily threaded to allow the mounting of a fluidic fitting. Via the bore 15, a fluid auxiliary medium, e.g. an exemplary liquid medium, e. g. water or Ringer's solution, or a gaseous auxiliary medium, e. g. water steam, is provided as auxiliary medium in operation. A circumferential recess 16 is provided on the outer wall surface of the inner cartridge element 11 in axial alignment with the bore 15.

From the circumferential recess 16, a number of exemplary four axial recesses 17a extend towards the cartridge bottom 1b that are for example equally distributed in circumferential direction. At the transition from the inner cartridge main section 11a to the inner cartridge bottom section 11b, each of the axial recesses 17a merges into an associated transversal recesses 17b that is arranged in the outer wall surface of the inner cartridge bottom section 11b. The transversal recesses 17b each run along a radius in a straight line from the periphery towards the center.

As best visible in FIG. 1e, a circumferential flow channel 18 is present between the bottom surface 11c of the inner cartridge element 11 respectively its bottom section 11b and the adjacent inner surface 12c of the outer cartridge element 12. The traversal recesses 17b run into the circumferential flow channel 18.

Exemplary three auxiliary apertures in form of bores 19 extend in the outer cartridge bottom section 12b from the circumferential flow channel 18 to the cartridge bottom 1b. The bores 19 exemplarily extend parallel to the cartridge axis and are arranged along a concentric arc around the nozzle 13 in an angle of exemplarily 90° (ninety degrees).

Via the circumferential flow channel 18, the transversal recesses 17b, the axial recesses 17a and the circumferential recess 16, the bores 19 as auxiliary apertures are fluidic coupled with the radial bore 15 as inlet opening, such that auxiliary medium that is provided via the radial bore 15 as inlet opening is distributed to the bores 19 as auxiliary apertures. The arrangement of circumferential flow channel 18, the transversal recesses 17b, the axial recesses 17a and the circumferential recess 16 accordingly serves as auxiliary medium distribution ductwork.

A material release piston (not shown) may be arranged inside the material reservoir 14 in a sealing and disposable manner to exert a force onto the tissue substitute material, thereby releasing tissue substitute material out of the nozzle 13 in a syringe-like way.

When using in the tissue substitute printing system, the cartridge bottom 1b faces the target where the tissue substitute is manufactured by way of printing. Tissue substitute material is released via the nozzle 13 and auxiliary medium is released via the bores 19 as auxiliary apertures onto the target, on the target, the paste-like tissue substitute material reacts with the auxiliary medium, resulting in the tissue substitute material setting.

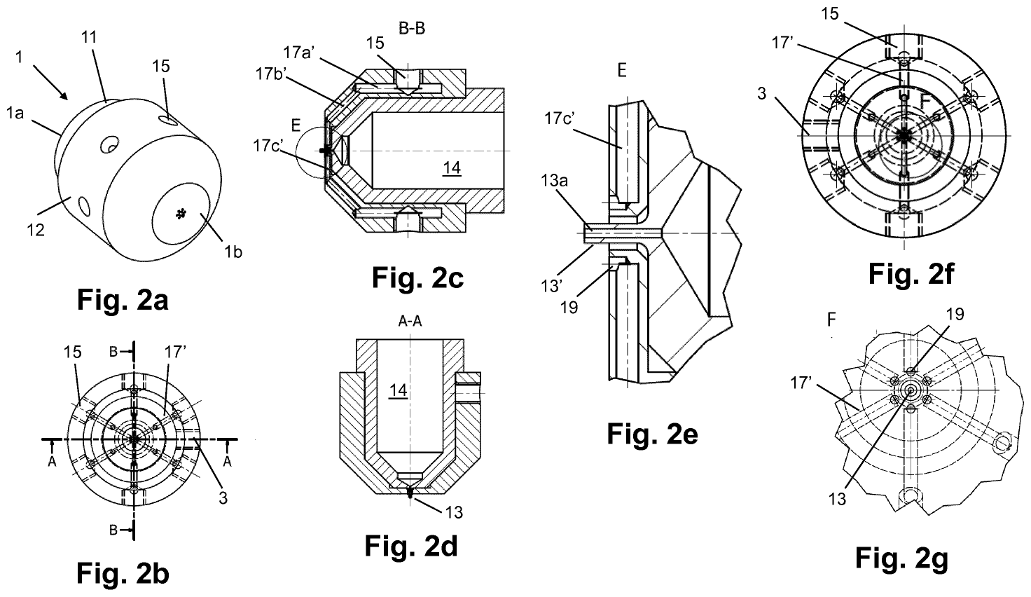

FIGS. 2a, to 2g show a further embodiment a cartridge 1. FIG. 2a shows a perspective view, FIG. 2b a top view and FIG. 2f a bottom view. FIG. 2c, and FIG. 2d are cross sectional views B-B and A-A as indicated in FIG. 2b. FIG. 2e shows detail E as indicated in FIG. 2c and FIG. 2g shows detail F as indicated in FIG. 2f.

Regarding a number of aspects, the embodiment as shown in FIGS. 2a to 2g is identical or similar to the embodiment as shown in FIG. 1a to 1g. The following description is focused on the differences.

The cartridge 1 of this embodiment is in particular different with respect to the release of the auxiliary medium. As best visible in FIG. 2g, exemplary six bores 19 as auxiliary apertures 19 are provided and arranged at the cartridge bottom 1b along a circle concentrically with a hollow needle element 13′, the bottom opening of which is the material aperture 13a. The auxiliary aperture bores 19 are exemplarily circumferentially equally distributed.

For each auxiliary aperture bore 19 an associated inlet opening in form of a bore 15 is present. Each inlet opening bore 15 is fluidic coupled with the associated auxiliary aperture bore 19 in a direct and unbranched way by way of an associated flow channel 17′, resulting in a one-to-one coupling between inlet opening bore 15 and associated auxiliary aperture bore 19.

The flow channels 17′ are embedded in the wall forming the outer cartridge element 1. Each flow channel 17′ comprises an axial flow channel section 17a′, that extends from the associated inlet opening bore 15 and runs axially towards the cartridge bottom 1b, followed, by transversal flow channel section 17b′ and finally a radial flow channel section 17c′ from which the associated auxiliary aperture bore 19 extends.

In contrast to the embodiment of FIGS. 1a-to 1g, this embodiment allows individual control of the auxiliary medium release via the auxiliary aperture bores 19, in dependence of the motion direction of the cartridge during the tissue substitute manufacturing process.

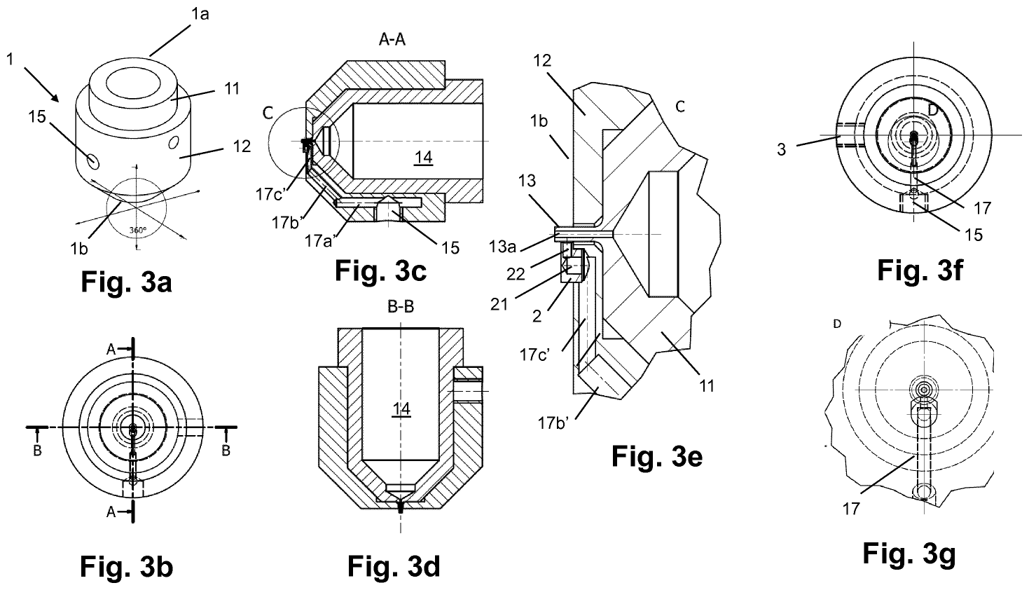

FIGS. 3a, to 3g show a further embodiment a cartridge 1. FIG. 3a shows a perspective view, FIG. 3b a top view and FIG. 3f a bottom view. FIG. 3c, and FIG. 3d are cross sectional views A-A and B-B as indicated in FIG. 3b. FIG. 3e shows detail C as indicated in FIG. 3c and FIG. 3g shows detail D as indicated in FIG. 3f.

In this embodiment, a single flow channel 17 is present for the auxiliary medium, which is exemplarily arranged similar to either of the flow channels 17′ in the embodiment of FIGS. 2a to 2g.

In this embodiment, the radial flow channel section 17c′ does not directly open into the auxiliary medium aperture. Instead, an auxiliary member 2 is arranged at the cartridge bottom 1b and inserted into a corresponding cutout of the outer cartridge element 12.

The auxiliary member 2 includes an axial flow channel element 21 and a radial flow channel element 22, both of which are realized as intersecting blind holes traverse to each other.

The radial flow channel section 17c′ opens into and thereby fluidic couples with the axial flow channel element 21. The radial flow channel element 22 opens towards the hollow needle element 13′ and is traverse to the hollow needle element 13′. The (non-referenced) opening of the radial flow channel element forms an auxiliary medium aperture which is in this embodiment traverse to the material aperture 13a.

Like in the embodiment of FIG. 2, a further radial threaded bore is present in the outer cartridge element 12 that is used for clamping and thereby fixing the inner cartridge element 11 via screw. In this way, one and the same outer cartridge element 12 may be used together with a number of inner cartridge element in series, i. e. the outer cartridge element may be re-used. This type of assembly, however, is not essential and other types of assembly, e. g. via a tight fit, may be used as well.

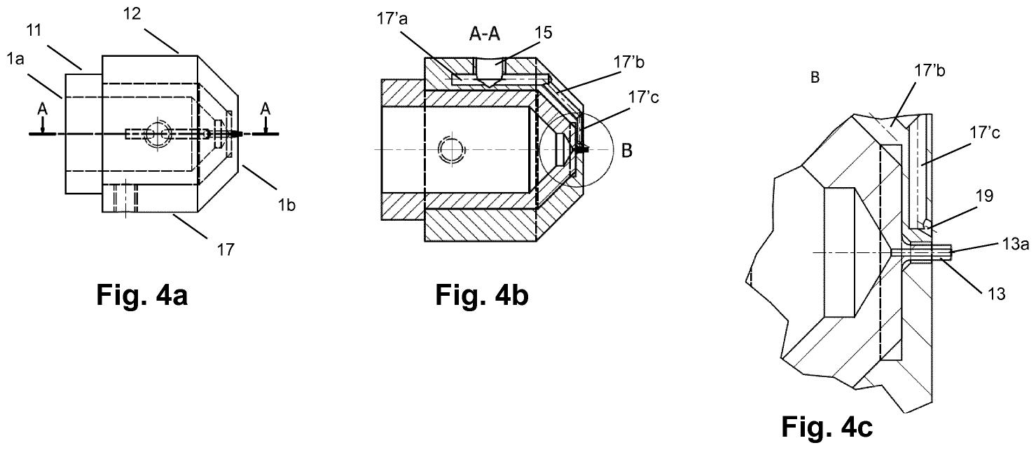

FIGS. 4a to 4c show a further embodiment of the cartridge 1. FIG. 4a shows a side view, FIG. 4b shows cross sectional view A-A as indicated in FIG. 4a, and FIG. 4c shows detail B as indicated in FIG. 4b.

In a number of aspects, this embodiment is similar to the before-described embodiments with the following description focusing in the differences. In this embodiment, a single flow channel 17 is present for the auxiliary medium, which is exemplarily arranged similar to the embodiment of FIGS. 3a to 3g. In contrast to the embodiment of FIGS. 3a to 3g, however, no auxiliary member is present. Instead, the radial flow channel 17c directly opens into and accordingly fluid couples with the auxiliary aperture bore 19, similar to the embodiment shown in FIGS. 2a to 2g. In the embodiment of FIGS. 4a to 4c, however, the auxiliary aperture bore is not arranged parallel to the hollow needle element 13′, but angled towards the hollow needle element 13′.

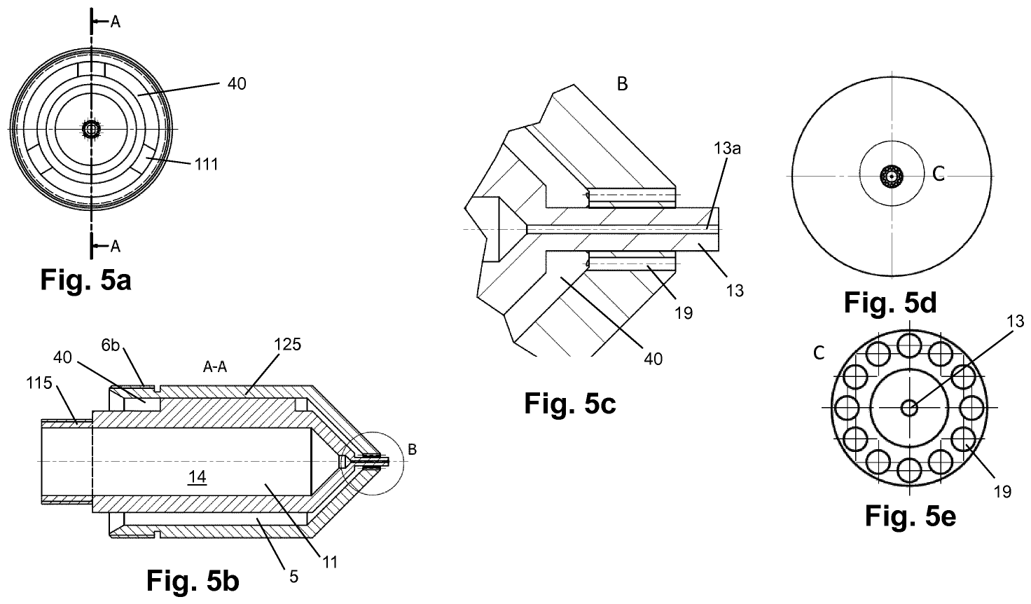

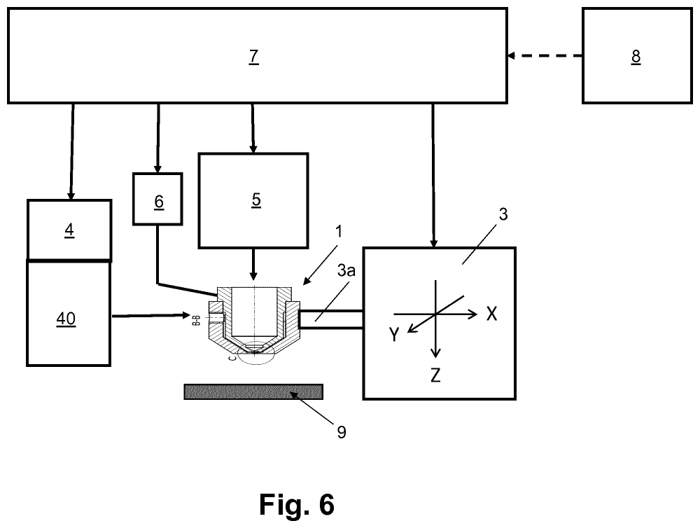

FIGS. 5a to 5e show a further embodiment of the cartridge 1. Figures a and 5d show a top view and a bottom view, respectively. FIG. 5b shows a sectional view along the line A-A as indicated in FIG. 5a. FIG. 5c shows an enlarged view of detail B as indicated in FIG. 5b and FIG. 5e shows an enlarged view of detail C as indicated in FIG. 5d.

The embodiment of FIG. 5a to FIG. 5e is different from the before-described embodiments in so far as the cartridge 1 includes an auxiliary medium reservoir 40 in addition to the material reservoir 14. The auxiliary medium reservoir 40 is realized by an annular gap between the outer circumferential surface of the inner cartridge element 11 and the outer cartridge element 12. The auxiliary medium reservoir 40 is of annular shape and circumferential surrounds the inner cartridge element 11.

For positioning and centering the inner cartridge element 11 within the outer cartridge element 12, a number of exemplarily four positioning elements in form of axial ribs 111 are present that extend radially from the generally cylindrical outer surface of the inner cartridge element 11. In assembly, the inner cartridge element 11 with the ribs 111 is axially inserted into the outer cartridge element 12, such that the ribs 111 contact the cylindrical inner surface of the outer cartridge element. The inner cartridge element 11 and the outer cartridge element 12 may be connected e. g. by gluing or tight via the ribs 111.

The ribs 111 do not extend axially along the whole length of the inner cartridge element 11, thereby ensuring that the auxiliary medium reservoir 40 is fluidic through-going or continuous. If desired, traverse channels, bores or recesses may be present that fluidic couple the compartments that are formed by the ribs 111 the ribs four in order ensure a continuous fluidic room.

As best visible in FIGS. 5c, 5e, the auxiliary medium reservoir 40 opens into a plurality of exemplarily 12 auxiliary aperture bores 19 which are arranged equally distributed along a circle in the center of which the hollow needle element is arranged.

Further in this embodiment, an exemplarily outer-threaded pressure supply fitting 115 is arranged in a top portion 112 of the inner cartridge element 11. In operation, the pressure supply fitting 115 is connected with a pressure supply, typically a gas pressure supply, for pressurizing the paste-like tissue substitute material within the material reservoir 14.

Similarly, a further outer-threaded pressure supply fitting 125 is arranged in a top portion of the outer cartridge element 12. In operation, the further pressure supply fitting 125 is connected with a pressure supply, typically a gas pressure supply, for pressurizing the auxiliary medium paste-like tissue substitute material within the auxiliary medium reservoir 40. A similar arrangement may be used in the other before-described embodiments as well.

In the following, reference is additionally made to FIG. 6. FIG. 6 shows an exemplary embodiment of a tissue substitute printing system together with a cartridge. in in a schematic functional view.

The cartridge 1 may generally be a cartridge according to any disclosed embodiment. The tissue substitute printing system includes a cartridge receptacle 3a. The cartridge receptacle receives the cartridge 1 in a replaceable manner. The cartridge receptacle 3a is operatively coupled to kinematic structure that is realized by a motorized x-y-z stage 3 for displacing the cartridge receptacle 3a with the cartridge 1 relative to a target receptacle 9. The target receptacle 9 is designed to receive a target on which the tissue substitute is manufactured.

The tissue substitute printing system further includes a material release controller 5 that is exemplarily realized by a controlled over pressure supply in fluidic coupling with the material reservoir (not referenced) of the cartridge 1 to pressurize the tissue substitute material inside the cartridge 1 via air pressure. Alternatively, the material release controller may be a spindle drive in operative coupling with a material release piston of the cartridge 1.

The tissue substitute printing system further includes an auxiliary medium release controller 4 that is exemplarily realized as over pressure supply, e. g. a compressor, to pressurize auxiliary medium, exemplarily water or Ringer solution, in an auxiliary medium reservoir 40 in fluidic coupling with the at least one auxiliary aperture of the cartridge 1 as explained before.

The tissue substitute printing system further includes an optional temperature controller 6 that is realized by a resistive or Peletier element and corresponding control circuitry, as discussed above in the general description.

The tissue substitute printing system further includes a computerized control unit 7 that controls operation of the material release controller 5, the auxiliary medium release controller 4, the x-y-z-stage 3, and the temperature controller 6. The control unit 7 may be based on a general-purpose computer running corresponding software code on one or more microprocessors, and/or on dedicated circuitry.

The control unit 7 is shown in operative coupling with an external computer 8 via which manufacture data for a tissue substitute are receives, e. g. CAD/CAM data. Optionally, the functionality of the external computer 8 may be integral with the control unit 7.

It is noted that the figures are partly simplified in order to focus on relevant aspects and may not show any detail. For example, the detailed design of the drug cartridge 1 may, in a practical embodiment, somewhat more complex. In particular, blind or embedded fluidic channels may, in dependence of the manufacturing technology and material used, be realized by machining, e. g. drilling, with non-required openings being subsequently closed by a plug.

### REFERENCE SIGNS

1 cartridge
1a cartridge top
1b cartridge bottom
11 inner cartridge element
11a inner cartridge main section
11b inner cartridge bottom section
11c bottom surface
12 outer cartridge element
12a outer cartridge main section
12b outer cartridge bottom section
12c inner surface
13 nozzle
13′ hollow needle element
13a material aperture
14 material reservoir
15 inlet opening/bore
16 circumferential recess
17a axial recess
17b transversal recess
17 flow channel
17a′ axial channel section
17b′ transversal flow channel section
17c′ radial flow channel section
18 circumferential flow channel
19 auxiliary aperture/bore
111 rib
112 top portion
113 shoulder
115 pressure supply fitting
125 further pressure supply fitting
2 auxiliary member
21 axial flow channel element
22 radial flow channel element
3 stage/kinematic structure
3a cartridge receptacle
4 auxiliary medium release controller
40 auxiliary medium reservoir
5 material release controller
6 temperature controller
7 control unit
8 external computer
9 target receptacle

1. A cartridge for use in a tissue substitute printing system, the cartridge comprising:
a material reservoir, the material reservoir storing a tissue substitute material in a flowable form;
a material aperture arranged at a bottom side of the cartridge, the material aperture being fluidic coupled with the material reservoir for releasing the tissue substitute material stored in the material reservoir; and
at least one auxiliary aperture arranged at the bottom side of the cartridge for releasing an auxiliary medium, the at least one auxiliary aperture being arranged in proximity to and fluidic separate from the material aperture.

2. The cartridge according to claim 1, wherein the cartridge comprises an auxiliary medium reservoir, the auxiliary medium reservoir storing an auxiliary medium, the auxiliary medium reservoir being fluidic coupled with the at least one auxiliary aperture.

3. The cartridge according to claim 2, wherein the auxiliary medium reservoir at least partially surrounds the material reservoir.

4. The cartridge according to claim 1, wherein the cartridge comprises a plurality of auxiliary apertures.

5. The cartridge according to claim 4, wherein the auxiliary apertures are arranged along an arc around the material aperture.

6. The cartridge according to claim 4 wherein the cartridge further comprises an auxiliary medium distribution ductwork, the auxiliary medium distribution ductwork being fluidic coupled with the plurality of auxiliary apertures.

7. The cartridge according to claim 1, wherein the cartridge comprises at least one inlet opening, the at least one inlet opening being fluidic coupled with the at least one auxiliary aperture.

8. The cartridge according to claim 7, wherein the at least one auxiliary aperture is fluidic coupled with an associated inlet opening via a point-to-point coupling.

9. The cartridge according to claim 1, wherein the cartridge comprises an inner cartridge element and an outer cartridge element, the outer cartridge element being arranged circumferentially around the inner cartridge element, wherein the inner cartridge element forms the material reservoir, wherein a flow channel in fluidic coupling with the at least one auxiliary aperture is arranged in an interface region between the outer cartridge element and the inner cartridge element.

10. The cartridge according to claim 1, wherein the auxiliary medium is either of water, an aqueous solution, a body fluid, cell culture liquid, Ringer's solution, a natural hydrogel, or a synthetic hyhydrogel.

11. The cartridge according to claim 1, wherein the cartridge includes a temperature controller, the temperature controller being arranged for controlling a temperature of the tissue substitute material in the material reservoir.

12. The cartridge according to claim 1, wherein the material aperture is a bore that is arranged in a cartridge bottom wall or a hollow needle element that projects beyond a cartridge bottom.

13. The cartridge according to claim 1, wherein the tissue substitute material comprises calcium phosphate.

14. A tissue substitute printing system, comprising:
a cartridge receptacle, the cartridge receptacle being configured to replaceable receive a cartridge according to claim 1;
a target receptacle;
a kinematic structure, the kinematic structure being configured to spatially displace the cartridge receptacle and the target receptacle relative to each other;
a material release controller, the material release controller being designed to control release of tissue substitute material via the material aperture;
an auxiliary medium release controller, the auxiliary medium release controller being designed to control release of auxiliary medium via the at least one auxiliary aperture; and
a computerized control unit, the control unit being configured to control operation of the material release controller and the auxiliary medium release controller for a metered release of tissue substitute material and a simultaneous or alternating release of auxiliary medium, and to simultaneously control the kinematic structure to displace the cartridge receptacle and the target receptacle relative to each other in accordance with a pre-determined tissue substitute geometry.

15. A method for manufacturing a tissue substitute, the method comprising:
releasing tissue substitute material in a flowable form from a cartridge in a controlled and metered manner onto a target;
simultaneously or alternatingly releasing an auxiliary medium from the cartridge, such that the material and the auxiliary medium react on the target; and
simultaneously spatially displacing the target and the cartridge with respect to each other in accordance with a pre-determined tissue substitute geometry.
